(19) United States
(12) Patent Application Publication
(10) Pub. No.: US 2009/0169658 A1
(43) Pub. Date: Jul. 2, 2009

(54) TOONA SINENSIS EXTRACT FOR SUPPRESSING PROLIFERATION AND INDUCING APOPTOSIS OF OSTEOSARCOMA CELLS

(75) Inventors: Mei-Ling Ho, Kaohsiung City (TW); Hseng-Kuang Hsu, Kaohsiung City (TW); Gwo-Jaw Wang, Kaohsiung City (TW); Je-Ken Chang, Kaohsiung City (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: KAOHSIUNG MEDICAL UNIVERSITY, Kaohsiung City (TW)

(21) Appl. No.: 11/965,795

(22) Filed: Dec. 28, 2007

(51) Int. Cl. A61K 36/58; C12N 5/00; A61P 35/04

(52) U.S. Cl. 424/761; 435/375

(57) ABSTRACT

Toona sinensis extract for suppressing the proliferation and inducing apoptosis of osteosarcoma, but not normal human osterblasts. The extraction process comprises: extracting Toona sinensis with water to obtain a first extract, and filtering the first extract by a membrane to obtain a filtrate, and the Toona sinensis extract of the invention does not cause biological damages of normal bone cells. In addition, the invention further provides a pharmaceutical composition comprising the Toona sinensis extract.

### BACKGROUND OF THE INVENTION

1. Field of the Invention

The present invention relates to an extract of Toona sinensis, and in particular, relates to an extract from the leaves of Toona sinensis for treating osteosarcoma.

2. Description of the Related Art

Toona sinensis or Cedrela sinensis, commonly known as Chinese mahogany cedar or Chinese Toona, is a perennial deciduous tree belonging to the Meliaceae plant family. Its bark is reddish brown. Its leaves are tender, edible and available almost all year around. Originally grown in the south-eastern part, the south-western part and the northern part of China, the Toona sinensis tree is now being grown in many countries. (Jennifer M. Edmonds and Martin Staniforth, TOONA SINENSIS (Meliaceae), Curtis's Botanical magazine, 15 (3), 186-193, 1998; Xiao-Dong Luo et al., Fitoterapia, 71, 492-496, 2000; Jong-Cheol Park et al., Kor. J. Pharmacogn, 27(3), 219-223, 1996).

Because the entire the Toona sinensis tree can be utilized, economic value is fairly high. According to reports almost every part of the Toona sinensis tree, including seeds, bark, root bark, petioles, and leaves, has medicinal effect (Jennifer M. Edmonds and Martin Staniforth, 1998, supra; Jong-Cheol Park et al., 1996, supra).

Seeds of the Toona sinensis tree contain oil, which is colorless and fragrant and can be used as edible oil. Shoots and leaves of the Toona sinensis tree are rich in carotene, amino acids and vitamins, and are therefore quite popular as a vegetable. In addition, mellowed leaves can be used as animal fodder.

According to literature (Jennifer M. Edmonds and Martin Staniforth, 1998 supra; Xiao-Dong Luo et al., 2000, supra), the bark, root bark and seeds of the Toona sinensis tree are useful in the treatment of neuralgia, duodenal ulcer, stomach upsets, gonorrhea, menstrual disorder, ascariasis, rheumatoid arthritis, and cancer, and are useful as an astringent, a carminative, an analgesic, and in suppressing growth of typhoid bacillus and amoeba protozoa (Si-Ming Yu and Ze-Dang Zhang, Journal of Anhui University Natural Science Edition No. 4, 91-94, 1990; Yue-Zhen Liu and Yu-Ping Li, Hebei Forestry Technology, No. 4, 51-52, December 1997).

According to literature, leaves of the Toona sinensis tree have anti-inflammatory, antidoting and worm-killing effects, and are useful for treating enteritis, dysentery, carbuncles, boils, dermatitis rhus, scabies, and tinea blanca, as well as for improving bodily health. In addition, aqueous extracts of leaves of the Toona sinensis tree have been used as a folk medicine for improving hypertension and diabetes. Hseng-Kuang Hsu et al. found that aqueous extracts of the Toona sinensis leaves are capable of lowering blood sugar in alloxan-induced diabetic rats (Wang P H et al., Toona sinensis increase GLUT4 glucose transporter protein in adipose tissue from Alloxan-induced diabetic rats, Annual Conference of Biomedical Science, p. 198, 2001). In another study, it was found that aqueous extracts from leaves of the Toona sinensis tree are capable of suppressing proliferation of human lung adenocarcinoma cells A549 (Hui-Chiu Chang et al. (2002), American Journal of Chinese Medicine, Vol. 30, Nos. 2 & 3, 307-314).

However, no prior art teaches or suggested a new use for the Toona sinensis or extracts for suppressing growth of osteosarcoma cells or treatment of osteosarcoma.

### BRIEF SUMMARY OF INVENTION

The invention provides a method for suppressing proliferation of osteosarcoma cells, comprising administrating an effective amount of the Toona sinensis extract, wherein the Toona sinensis extract is extracted with water.

The invention provides a method for suppressing proliferation of osteosarcoma cells, comprising administrating an effective amount of the Toona sinensis extract, wherein wherein the Toona sinensis extract is prepared by the steps of: extracting Toona sinensis with water to obtain a first extract; filtering the first extract by a 30 to 100 mesh membrane to obtain a filtrate; centrifuging the first extract to obtain a supernatant, and lyophilizing the supernatant.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIGS. 1a-1b show that the Toona sinensis extract of the invention suppresses the proliferation of U2-OS and Saos-2 cells;

FIGS. 2a-2b show that the sterilized extract of Toona sinensis has a better suppression efficiency of osteosarcoma cells than unfermented extract;

FIG. 3 shows the extract of Toona sinensis does not cause biological damages of normal bone cells;

FIG. 4 shows that the cell cycle of osteosarcoma cells is arrested at the G₂ phase after treatment of the Toona sinensis extract;

FIG. 5 shows that the extract of Toona sinensis induces the expression of p21, p-cdc25C RNA, but represses the expression of cdc-2, and cyclin B1 RNA;

FIG. 6 shows that the Toona sinensis extract of the invention induces the expression of p21, p-cdc25C protein, but represses the expression of cdc-2, and cyclin B1 protein in osteosarcoma cell;

FIG. 7 shows that the Toona sinensis extract of the invention induces the release of LDH (lactate dehydrogenase) in osteosarcoma cells;

FIG. 8 shows that the Toona sinensis extract of the invention induces the apoptosis of osteosarcoma cells;

FIGS. 9a-9b show that the Toona sinensis extract of the invention suppresses the RNA expression of Bcl-2 gene, but induces the RNA expression of Bax gene;

FIGS. 10a-10b show that the Toona sinensis extract of the invention suppresses the protein expression of Bcl-2 gene, but induces the protein expression of Bax gene;

FIG. 11 shows that the PARP protein is degraded from 116 kDa to 85 kDa by the Toona sinensis extract of the invention; and

FIG. 12 shows that the Toona sinensis extract of the invention increases the expression of Cyclophilin A proteins in U2-OS, Saos-2, and MG-63 cells.

### DETAILED DESCRIPTION OF INVENTION

The following description is of the best-contemplated mode of carrying out the invention. This description is made for the purpose of illustrating the general principles of the invention and should not be taken in a limiting sense. The scope of the invention is best determined by reference to the appended claims.

The invention provides a method for preventing proliferation and inducing apoptosis of osteosarcoma cells, comprising administrating an effective amount of the Toona sinensis extract, wherein the Toona sinensis extract is extracted with water.

The Toona sinensis extract of the invention can be extracted from the new leaves or tender buds of Toona sinensis, preferably, new leaves.

The extraction method of the Toona sinensis extract includes: (a) extracting the leaves of the Toona sinensis with water by heating to obtain a first extract; (b) filtering the first extract by a membrane to obtain a filtrate; (c) centrifuging the first extract to obtain a supernatant; and (d) lyophilizing the supernatant to obtain the Toona sinensis extract.

Firstly, a suitable amount of water is added to the leaves or tender buds of the Toona sinensis, and heated to a boil and kept boiling. Then, the leaves are removed and filtered with a 30-100 mesh filter sieve, preferably, a 70 mesh filter sieve, to obtain a filtrate. The filtrate is centrifuged at 4° C. at 2000 to 4000 rpm for 8 to 15 minutes to obtain a supernatant.

In one embodiment, the Toona sinensis extract is further processed by stem sterilization (autoclave sterilization). The condition of the stem sterilization can be 100 to 130° C. for 10 to 20 minute, preferably, 121° C. for 15 minutes.

In another embodiment, the Toona sinensis extract is further processed by the steps of: loading the Toona sinensis extract onto a liquid chromatography column; eluting the reverse phase column with an alcohol solution; and collecting the alcohol eluate.

In another embodiment, the Toona sinensis extract is further processed by the steps of: extracting the Toona sinensis extract with an alcohol solution to obtain a second extract; centrifuging the first extract to obtain a supernatant, and lyophilizing the supernatant.

The alcohol of the invention includes, but is not limited to, methanol, ethanol, propyl alcohol, isopropanol, n-butanol, iso-butanol, or a combination thereof.

The Toona sinensis extract of the invention can effectively inhibit the proliferation of cancer cells, preferably, osteosarcoma cells, such as, U2-OS, MG-63, or Saos-2, etc. The Toona sinensis extract of the invention can induce p21, p35 or p-cdc25 protein expression and suppress cdc-2 or cyclin B1 protein expression in the osteosarcoma cells to arrest the cell cycle of osteosarcoma cells at G₂ phase.

Additionally, the Toona sinensis extract of the invention relates to the apoptosis of osteosarcoma cells. The Toona sinensis extract of the invention can induce the expression of Bax and Cyclophilin A protein and suppress the expression of Bcl-2 protein in the osteosarcoma cells to induce the apoptosis of the osteosarcoma cells.

The composition of the invention prevents and/or treats cancer so that the composition can be administrated to cancer patients, chemotherapy patients, and high-risk group cancer patients, etc. In addition, the composition is very safe and does not cause biological damage so that the composition can also serve as a food supplement.

The invention further provides a pharmaceutical composition for preventing the growth of osteosarcoma cells. The composition of the invention comprises an effective amount of the Toona sinensis extract, and a pharmaceutically acceptable carrier or excipient.

The term “pharmaceutically acceptable carrier” as used herein refers to carriers known in the art to be suitable for the manufacturing of pharmaceuticals and including, but not limited to, water, normal saline, glycerin, organic solvents, stabilizers, chelating agents, preservatives, emulsifiers, suspending agents, diluents, gel-forming agents, liposomes, etc.

The pharmaceutical composition of the preset invention may be prepared by a method known in the art into forms suitable for parenteral, oral or topical administration, including, but not limited to, injection, solution, capsule, dispersion, suspension, etc.

To produce an oral solid preparation, an excipient and, if necessary, a binder, a disintegrator, a lubricant, a coloring matter, a flavoring agent and/or the like may be admixed with an extract of this invention. The resultant mixture can then be formed into tablets, coated tablets, granules, powder, capsules or the like by a method known per se in the art. Such additives can be generally employed in the present field of the art, including excipients: lactose, sucrose, sodium chloride, glucose, starch, calcium carbonate, kaolin, micro-crystalline cellulose, and silicic acid; binders: water, ethanol, propanol, sucrose solution, glucose solution, starch solution, gelatin solution, carboxymethylcellulose, hydroxypropylcellulose, hydroxypropylstarch, methyl-cellulose, ethylcellulose, shellac, calcium phosphate, and polyvinylpyrrolidone; disintegrators: dry starch, sodium alginate, powdered agar, sodium hydrogencarbonate, calcium carbonate, sodium lauryl sulfate, monoglycerol stearate, and lactose; lubricants: purified talc, stearate salts, borax, and polyethylene glycol; and corrigents: sucrose, bitter orange peel, citric acid, and tartaric acid.

To produce an oral liquid preparation, a flavoring agent, a buffer, a stabilizer and the like may be admixed with an extract of this invention. The resultant mixture can then be formed into a solution for internal use, a syrup, an elixir or the like by a method known per se in the art. In this case, the flavoring agent can be the same as that mentioned previously. The buffer can be a sodium citrate, while illustrations of the stabilizer are tragacanth, gum arabic, and gelatin.

To prepare an injection, a pH regulator, a buffer, a stabilizer, an isotonicity and the like may be admixed with a compound of this invention. The resultant mixture can then be formed into a subcutaneous, intramuscular or intravenous injection by a method known per se in the art. pH regulator and buffer include, and are not limited to, sodium citrate, sodium acetate, and sodium sulfate. The stabilizer includes sodium pyrosulfite, EDTA, thioglycollic acid, and thiolactic acid. The isotonicity includes sodium chloride and glucose.

The amount of active ingredients that can be combined with the carrier materials to produce a single dosage form will vary depending upon the subject and the particular mode of administration. The dosage required will vary according to a number of factors known to those skilled in the art, including, but not limited to, the compound or compounds used, the species of subject, the size of the subject, and the severity of the associated disease condition that causes pruritus. The compounds can be administered in a single dose, in multiple doses throughout a 24-hour period, or by continuous infusion. When administered by continuous infusion, the compounds can be supplied by methods well known in the art, such as, but not limited to, intravenous gravity drip, intravenous infusion pump, implantable infusion pump, or any topical routes. The subject can be treated with the extract of the Toona sinensis individually or in combination with other treatments, such as chemotherapy or radiotherapy.

### EXAMPLE

### Example 1

### Preparation of Extracts from Leaves of the Toona Sinensis

Tender leaves of the Toona sinensis were picked and washed briskly with water. A suitable amount of water was added to the leaves in a proportion of 4 liters of RO water to 1 kg of leaves. The mixture was heated to a boil and kept boiling. Then, the leaves were removed, and the remainder was heated slowly to a concentrate, which was filtered with a filter sieve (70-mesh). The filtered concentrate was lyophilized using a Virtis apparatus to obtain a crude extract, which was called “TSL-CE”.

Additionally, the filtered concentrate could be subjected to centrifugation prior to lyophilization. The filtered concentrate was centrifuged at 4° C. at 3000 rpm (Beckman Avanti™ J-30I) for 12 minutes to give a supernatant portion and a precipitate portion containing insoluble substances. The supernatant portion was subjected to lyophilization using a Virtis apparatus to obtain a lyophilized water extract, which was called “TSL-1”.

50 g of extract “TSL-1” obtained from the aforesaid extraction procedure A was dissolved in 99.5% ethanol to carry out alcohol extraction. The alcohol solution thus formed was centrifuged to give a supernatant portion and a precipitate portion. The supernatant portion was further subjected to lyophilization using a Virtis apparatus to obtain a further purified alcohol extract in the form of lyophilized powder, which was called “TSL-2”. The TSL-2 was subsequently dissolved in different concentration of ethanol (99.5%→50%→25%→12.5%). The ethanol solution thus formed was centrifuged at 4° C. and at 3000 rpm for 12 minutes to give a supernatant portion and a precipitate portion. The supernatant portion was further subjected to lyophilization using a Virtis apparatus to obtain an extract in the form of lyophilized powder, which was called “TSL-3, TSL-4, and TSL-5”. The TSL-2 was subsequently dissolved in water and centrifuged to obtain a supernatant portion. The supernatant portion was further subjected to lyophilization to obtain an extract in the form of lyophilized powder, which was called “TSL-6 and TSL-7”. The preparations of extracts from Leaves of the Toona sinensis are described in U.S. Pat. No. 7,229,652.

### Example 2

### Effect of the Toona Sinensis Extract on Inhibiting Proliferation of Osteosarcoma Cell

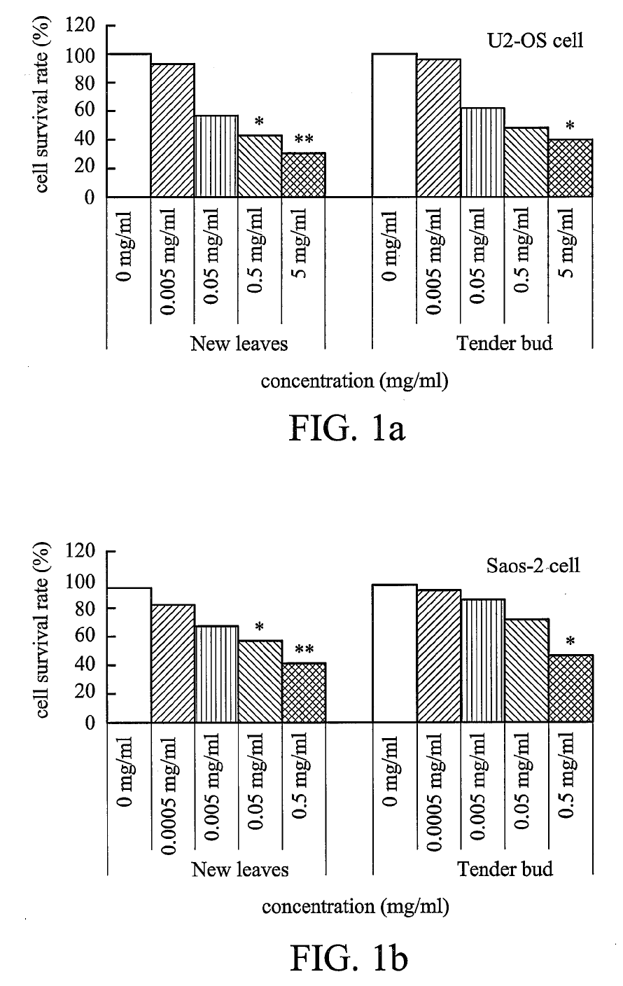

The effect of the Toona sinensis extract on the inhibition of cell proliferation of osteosarcoma cells was analyzed by an MTT assay. FIGS. 1A and 1B shows the MTT assay results of U2-OS cell and Saos-2 cell, respectively. Referring to FIGS. 1A-1B, the TSL-1 extracted from new leaves could effectively suppress the proliferation of U2-OS cell and Saos-2 cell (IC₅₀ was 59.6 μg/ml and 86.4 μg/ml for U2-OS cell and Saos-2 cell, respectively), and the TSL-1 extracted from tender buds could slightly suppress the proliferation of U2-OS cell and Saos-2 cell (IC₅₀ was 64.7 μg/ml and 98.2 μg/ml for U2-OS cell and Saos-2 cell, respectively).

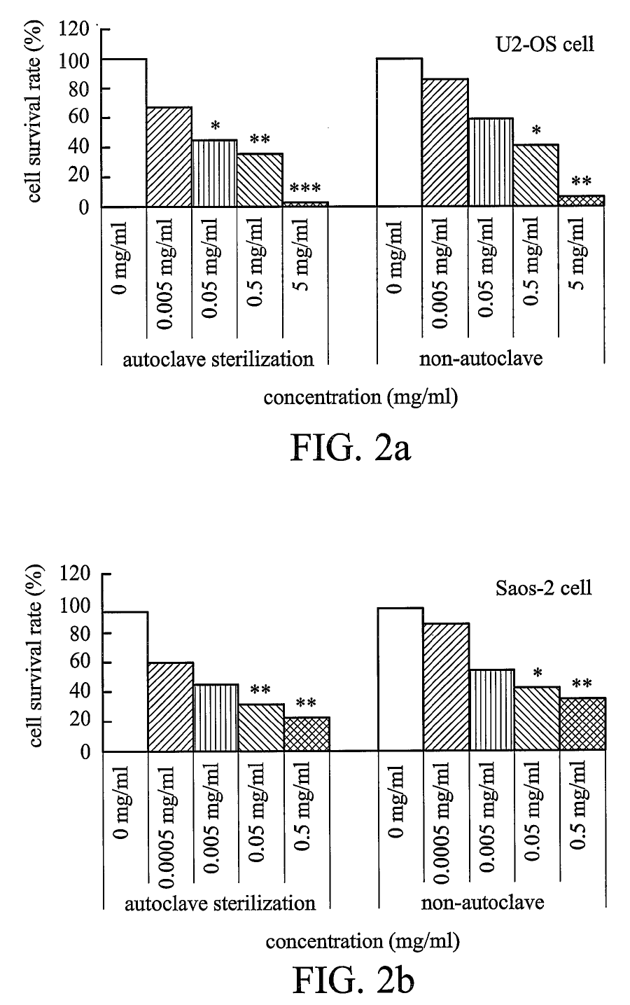

Additionally, TSL-1 was further treated by steam sterilization, and then the osteosarcoma cell (U2-OS cell and Saos-2 cell) was cultured on a medium containing the sterilized TSL-1 for 72 hours. In the control group, the TSL-1 was not treated with steam sterilization. FIGS. 2A and 2B shows the MTT assay results of U2-OS cell and Saos-2 cell, respectively. Referring to FIGS. 2A-2B, in comparison, the sterilized TSL-1 had a higher anti-osteosarcoma effect than that without sterilization. For example, IC₅₀ of the sterilized TSL-1 was 46.3 μg/ml and 39 μg/ml for U2-OS cell and Saos-2 cell, but IC₅₀ of the TSL-1 without sterilization was 59.8 μg/ml and 59 μg/ml for U2-OS cell and Saos-2 cell, respectively.

### Example 3

### Effect of the Toona Sinensis Extract on the Proliferation of Normal Bone Cell

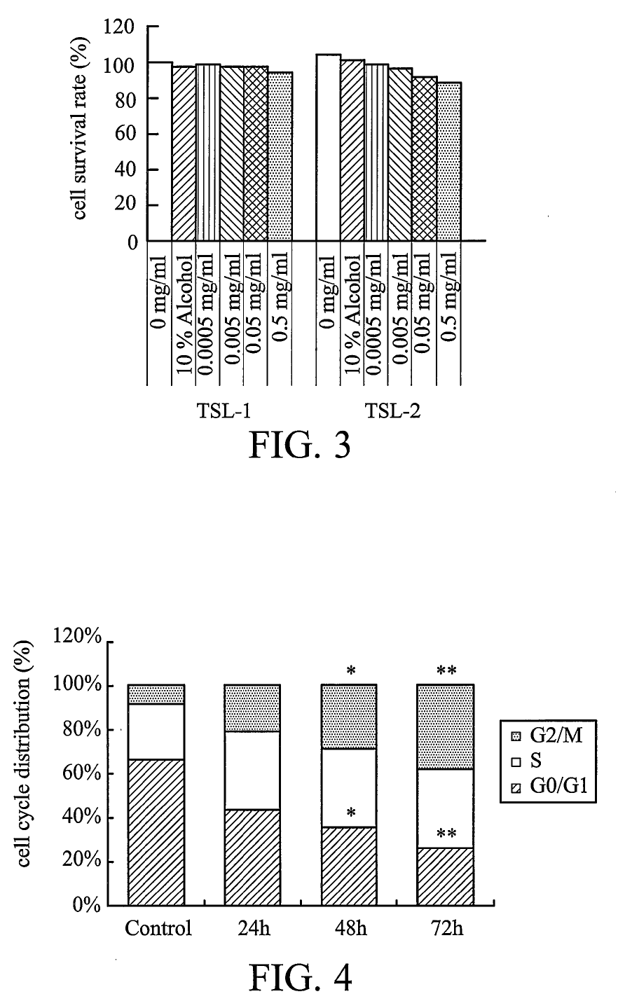

The human osteoblast cell was treated with TSL-1, TSL-2, and TSL-1-5-7 extracted from new leaves, respectively, to analyze the effect of the Toona sinensis extract on the growth of normal bone cells. The treatment time was 72 hours. Referring to FIG. 3, TSL-1 and TSL-2 did not suppress the proliferation of the human osteoblast cells. Accordingly, FIG. 3 indicates that TSL-1 not only suppressed the proliferation of osteosarcoma cells, but also did not cause biological damage to normal cell.

### Example 4

### Effect of TSL-1 on the Cell Cycle of Osteosarcoma Cell

An osteosarcoma cell line MG-63 was treated with a medium containing 0.05 mg/ml of TSL-1 for 24, 48, and 72 hours, respectively, and then analyzed by flow cytometry to determine the cell cycle distribution of the MG-63 cell. In the control group, the MG-63 cell was not treated with TSL-1. Referring to FIG. 4, after treatment of TSL-1, the MG-63 cell appeared to stay at G₂/M phase. The cell number at G₂ phase was 14.9%, 29.5, and 37.3% after treatment of TSL-1 for 24, 48 and 72 hours, respectively.

### Example 5

### Effect of TSL-1 on the RNA Expression of Cell Cycle Control Gene (Cyclin B1, Cyclin A, cdc2, p-cdc25C, p21, p53 and p27) in Osteosarcoma Cells

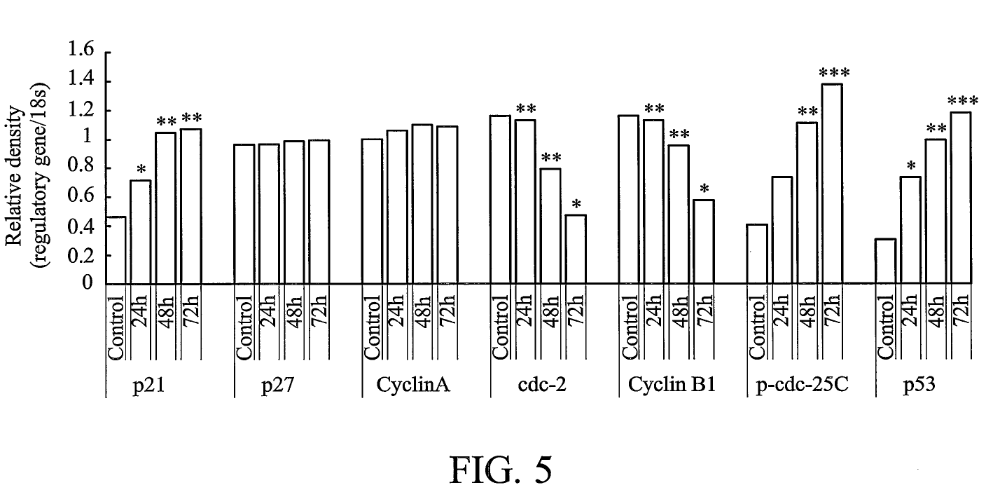

An osteosarcoma cell line MG-63 was treated with TSL-1, and then the RNA expression level of cyclin B1, cyclin A, cdc2, p-cdc25C, p21, p53 and p27 genes in MG-63 cell were analyzed by RT-PCR. In the control group, the MG-63 cell was not treated with TLS-1. Referring to FIG. 5, the RNA expression of p21, p53, p-cdc25C gene was increased dependent upon increasing treatment time of TSL-1, the RNA expression of cdc-2 and cyclin B1 was decreased dependent upon increasing treatment time of TSL-1, and the RNA expression of p27 and cyclin A did not changed. Accordingly, TSL-1 arrested the cell cycle of the MG-63 at G₂/M phase and effect the RNA expression of cdc-2, cyclin B1, p21, p53, p-cdc25C gene.

### Example 6

### Effect of TSL-1 on the Protein Expression of Cell Cycle Control Gene (Cyclin B1, Cyclin A, cdc2, p-cdc25C, p21, p53 and p27) in Osteosarcoma Cell

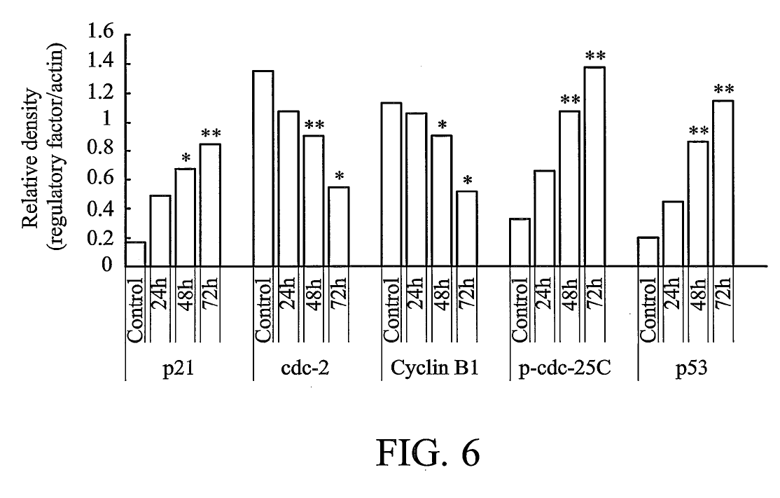

An osteosarcoma cell line MG-63 was treated with TSL-1, and then the protein expression level of cyclin B1, cyclin A, cdc2, p-cdc25C, p21, p53 and p27 gene in MG-63 cell was analyzed by western blot. In the control group, the MG-63 cell was not treated with TLS-1. Referring to FIG. 6, the protein expression of p21, p53, and p-cdc25C gene was increased dependent upon increasing treatment time of TSL-1, but the protein expression of cdc-2 and cyclin B1 was decreased dependent upon increasing treatment time of TSL-1. Accordingly, TSL-1 arrested the cell cycle of the MG-63 at G₂/M phase and effected the protein expression of cdc-2, cyclin B1, p21, and p-cdc25C genes.

### Example 7

### TSL-1 Induces Cytotoxicity of Osteosarcoma Cells

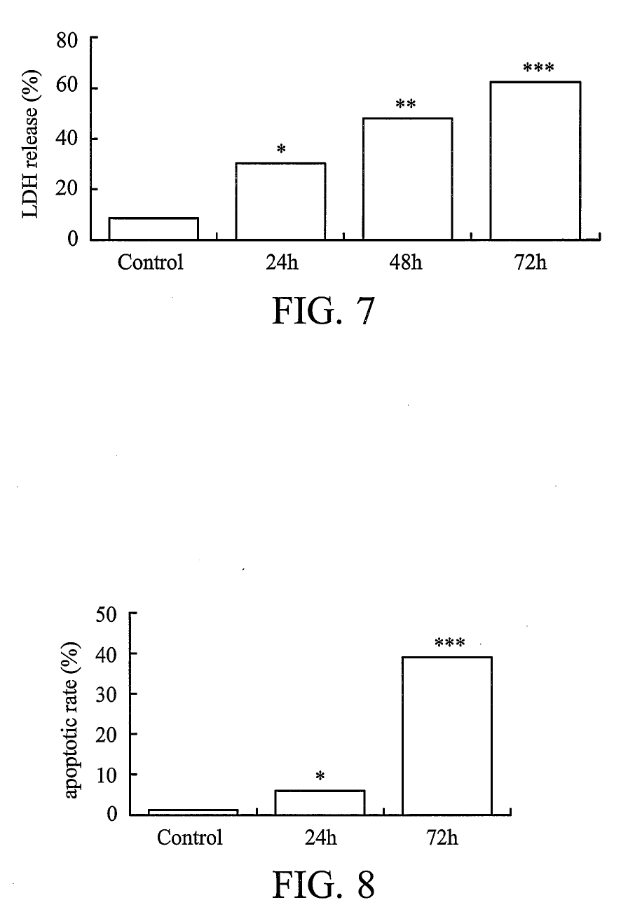

The effect of TSL-1 on an osteosarcoma cell line MG-63 was investigated by released amounts of lactate dehydrogenas (LDH). In the control group, the MG-63 cell was not treated with TLS-1. Referring to FIG. 7, after treatment of 0.05 mg/ml of TSL-1 for 24, 48, and 72, the LDH of the MG-63 cell was released, and the released amounts of LDH was associated with treatment time.

### Example 8

### TSL-1 Induces the Apoptosis of Osteosarcoma Cell Line MG-63

The apoptosis of the MG-63 was investigated by TUNEL assay. In the TUNEL stain, the apoptotic cell was a stained red color. In the control group, the MG-63 cell was not treated with TLS-1. Referring to FIG. 8, after treatment of 0.05 mg/ml of TSL-1 for 24, 48, and 72, the number of the apoptotic cells was increased, and the increase was dependent upon increasing treatment time of TSL-1.

### Example 9

### Effect of TSL-1 on RNA and Protein Expression of Bax and Bcl-2 Genes in Osteosarcoma Cells

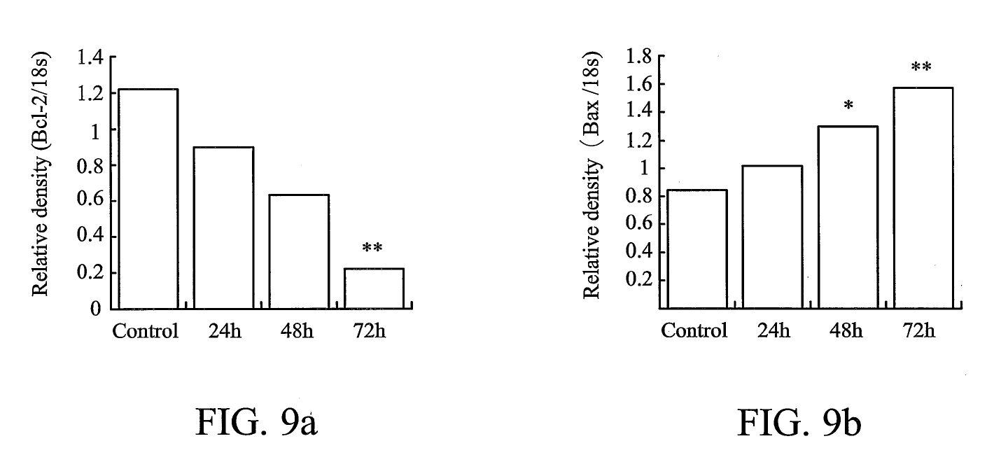

An osteosarcoma cell line MG-63 was treated with 0.05 mg/ml of TSL-1 for 24, 48, and 72, and then the RNA and protein expression of Bax and Bcl-2 gene in the MG-63 cell were analyzed by RT-PCR and western blot. FIG. 9A shows the RNA expression and proliferation status of Bcl-2 gene, and FIG. 9B shows the RNA expression and proliferation status of Bax gene. Referring to FIG. 9A-9B, the RNA expression of Bcl-2 gene was decreased dependent upon increasing of the treatment time of TSL-1 (The expression of Bcl-2 RNA decreased about 29%, 42%, and 75% when compared to the control group at 24, 48, and 72 hours, respectively). However, the RNA expression of Bax gene was increased dependent upon increasing of the treatment time of TSL-1 (The expression of Bax RNA increased about 10%, 23%, and 46% when compared to the control group at 24, 48, and 72 hours, respectively).

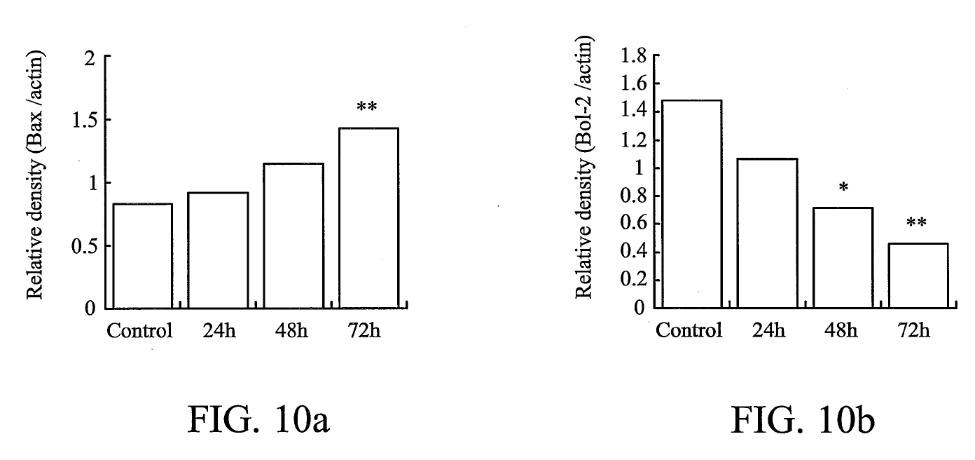

FIG. 10A shows the protein expression and proliferation status of Bcl-2 gene, and FIG. 10B shows the protein expression and proliferation status of Bax gene. Referring to FIG. 10A-10B, the protein expression of Bcl-2 gene decreased, dependent upon increasing of the treatment time of TSL-1 (The expression of Bcl-2 protein decreased 25%, 46%, and 75% when compared to the control group at 24, 48, and 72 hours, respectively). However, the protein expression of Bax gene increased dependent upon increasing of the treatment time of TSL-1 (The expression of Bax protein increased 9.5%, 23%, and 55% when compared to the control group at 24, 48, and 72 hours, respectively).

### Example 10

### TSL-1 Induces the Apoptosis of Osteosarcoma Cells

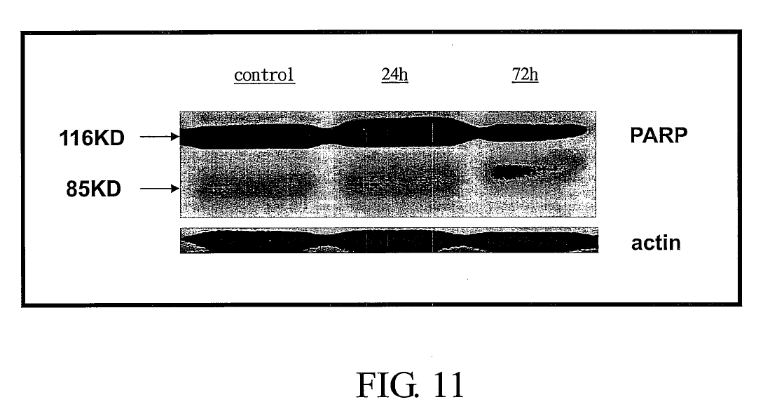

An osteosarcoma cell line MG-63 was treated with TSL-1 for 24, 48, and 72 hours, and then analyzed by western blot to investigate the degradation of poly (ADP-ribose) polymerase (PARP). In the control group, MG-63 cell was not treated with TLS-1. Referring to FIG. 11, PARP was degraded from 116 kDa to 85 kDa, and the degradation of PARP was obvious dependent upon increasing of the treatment time of TSL-1. Accordingly, TSL-1 induced the apoptosis of MG-63 cell.

### Example 11

### Effect of TSL-1 on Expression of Cyclophilin a Protein in an Osteosarcoma Cell Line U2-OS, Saos-2, and MG-63

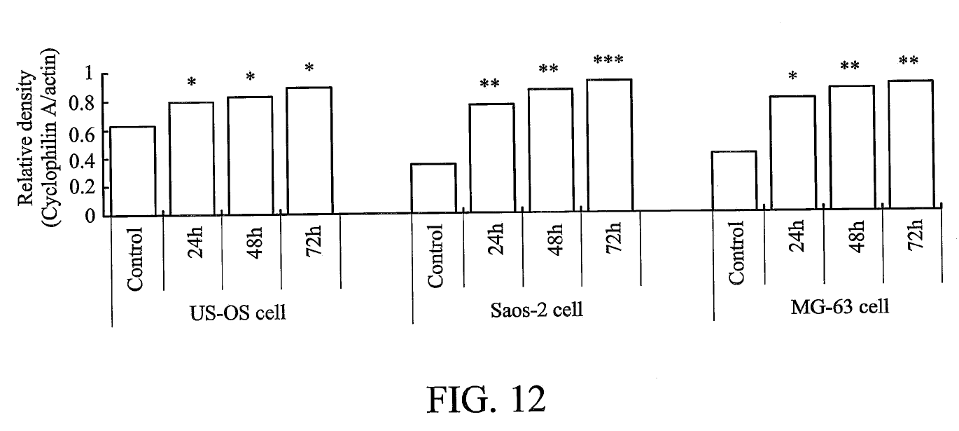

An osteosarcoma cell line U2-OS, Saos-2, and MG-63 were treated with 0.05 mg/ml of TSL-1 for 24, 48, and 72 hours, and then analyzed by western blot to investigate the expression of Cyclophilin A protein. In the control group, the U2-OS, Saos-2, and MG-63 cells were not treated with TLS-1. Referring to FIG. 12, after treatment of TSL-1, the expression of Cyclophilin A proteins in the U2-OS, Saos-2, and MG-63 cells increased, wherein the expression of Cyclophilin A proteins of the U2-OS was higher than that of Saos-2 and MG-63 cells (U2-OS cell: the expression of Cyclophilin A protein increased 75%, 100%, and 122.5% when compared to the control group at 24, 48, and 72 hours, respectively. Saos-2 cell: the expression of Cyclophilin A protein increased 33%, 41%, and 50% when compared to the control group at 24, 48, and 72 hours, respectively. MG-63 cell: the expression of Cyclophilin A protein increased 55%, 70%, and 78% when compared to the control group at 24, 48, and 72 hours, respectively.).

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for suppressing proliferation of osteosarcoma cells, comprising administrating an effective amount of the Toona sinensis extract, wherein the Toona sinensis extract is extracted with water.

2. The method as claimed in claim 1, wherein the Toona sinensis extract is prepared by the steps of:
extracting Toona sinensis with water to obtain a first extract; and
filtering the first extract by a 30 to 100 mesh membrane to obtain a filtrate.

3. The method as claimed in claim 2, wherein the first extract is further processed by the steps of:
centrifuging the first extract to obtain a supernatant, and
lyophilizing the supernatant to obtain the Toona sinensis extract.

4. The method as claimed in claim 3, wherein the Toona sinensis extract is further treated with a steam sterilization process.

5. The method as claimed in claim 3, wherein the Toona sinensis extract is further processed by the steps of:
loading the Toona sinensis extract onto a liquid chromatography column;
eluting the liquid chromatography column with an alcohol solution, and
collecting the alcohol eluate;

6. The method as claimed in claim 3, wherein the Toona sinensis extract is further processed by the steps of:
extracting the Toona sinensis extract with an alcohol solution to obtain a second extract;
centrifuging the second extract to obtain a supernatant, and
lyophilizing the supernatant.

7. The method as claimed in claim 1, wherein the Toona sinensis extract is extracted from the new leaves or tender buds.

8. The method as claimed in claim 5, wherein the alcohol comprises methanol, ethanol, propyl alcohol, isopropanol, n-butanol, iso-butanol, or a combination thereof.

9. The method as claimed in claim 6, wherein the alcohol comprises methanol, ethanol, propyl alcohol, isopropanol, n-butanol, iso-butanol, or a combination thereof.

10. The method as claimed in claim 1, wherein the Toona sinensis extract induces the expression of p21, p53, p-cdc25 and Bax protein in osteosarcoma cells.

11. The method as claimed in claim 1, wherein the Toona sinensis extract suppresses the expression of cdc-2, cyclin B1, and Bcl-2 protein in osteosarcoma cells.

12. The method as claimed in claim 1, wherein the Toona sinensis extract induces the apoptosis of osteosarcoma cells.

13. A pharmaceutical composition, comprising an effective amount of the Toona sinensis extract as claimed in claim 1, and a pharmaceutically acceptable carrier or excipient.

14. A method for suppressing proliferation of osteosarcoma cells, comprising administrating an effective amount of the Toona sinensis extract, wherein the Toona sinensis extract is prepared by the steps of:
extracting Toona sinensis with water to obtain a first extract;
filtering the first extract by a 30 to 100 mesh membrane to obtain a filtrate;
centrifuging the first extract to obtain a supernatant, and
lyophilizing the supernatant.
